(19) United States
(12) Patent Application Publication
(10) Pub. No.: US 2016/0244835 A1
(43) Pub. Date: Aug. 25, 2016

(54) URINARY NEUROPILIN-1 (NRP-1) AS A PROGNOSTIC MARKER FOR NEPHRITIS AND LUPUS NEPHRITIS

(71) Applicants: CONSEJO SUPERIOR DE INVESTIGACIONES CIENTÍFICAS (CSIC), Madrid (ES); FUNDACIÓ HOSPITAL UNIVERSITARI VALL D'HEBRON - INSTITUT DE RECERCA, Barcelona (ES)

(72) Inventors: Georgina HOTTER CORRIPIO, Barcelona (ES); Ana Maria SOLA MARTINEZ, Barcelona (ES); Jose Luis VINAS MUNOZ, Barcelona (ES); Josep ORDIROS, Barcelona (ES); Maria Teresa TORRES SALIDO, Barcelona (ES); Josefina CORTES HERNANDEZ, Barcelona (ES)

(73) Assignees: CONSEJO SUPERIOR DE INVESTIGACIONES CIENTIFICAS (CSIC), Madrid (ES); FUNDACIO HOSPITAL UNIVERSITARI VALL D'HEBRON - INSTITUT DE RECERCA, Barcelona (ES)

(21) Appl. No.: 14/903,716

(22) PCT Filed: Jul. 9, 2014
(86) PCT No.: PCT/ES2014/070564

§ 371 Date: Apr. 29, 2016

(30) Foreign Application Priority Data
Jul. 10, 2013 (ES) P201331051

(51) Int. Cl. C12Q 1/68; G01N 33/68

(52) U.S. Cl. CPC C12Q 1/6883; G01N 33/6893; C12Q 2600/158; C12Q 2600/106; C12Q 2600/118; G01N 2333/705; G01N 2800/347; G01N 2800/52

(57) ABSTRACT

This invention refers to the methods for predicting the progress of nephritis and lupus nephritis in an individual. This invention also refers to the methods for evaluating the development of nephritis, particularly lupus nephritis, in an individual, and his/her response to a treatment.

### SECTOR AND OBJECT OF THE INVENTION

The field of this invention is the Area of Biotechnology in the Pharmaceutical Industry sectors. Specifically, this invention refers to the use of Neuropilin-1 as a prognostic biomarker for nephritis and lupus nephritis in clinical practice.

### STATE OF THE ART

Systemic Lupus Erythematosus (SLE) is an autoimmune disease in which kidney damage is one of the determining factors in a bad prognosis (1). Lupus nephritis (LN) appears in approximately 40-75% of patients with SLE and is associated with an unpredictable course and an increase in early and late morbidity (2). Despite carrying out an initial aggressive treatment, up to 25% of patients with LN will progress to renal damage (3), where this is partially caused by the difficulty in evaluating early the response to the treatment. Having a predictive index would enable new treatments to be introduced which could modify the course of the disease.

At the moment, a renal biopsy is the “Gold Standard” for diagnosing and evaluating the response to treatment for renal damage, but because of its invasive nature, it cannot be carried out in a serial manner. Traditional serological biomarkers such as anti-DNA antibodies, complement levels and kidney function tests (proteinuria, urinary sediment, creatinine and glomerular filtration rate) have been shown to be insufficient (4, 5) and are not always correlated with kidney damage. Up until now, these diagnostic tools have not allowed us to predict reliably these patients' response to treatment until after the first 6 months of immunosuppressant treatment (6), and it is the consequences for the kidneys (renal fibrosis) that occur in this period which will determine the long-term prognosis (7).

For this reason, in recent years there has been increasing interest in finding non-invasive markers for lupus nephritis which can be used to predict the start of the illness and also monitor its progression. Urinary biomarkers, such as IL-12, TWEAK, MCP-1 or NGAL, are very attractive candidates (8, 9) given that, apart from being relatively easy to obtain, they can also reflect physiopathological changes in the kidneys. However, there needs to be a longitudinal validation in larger groups of patients with lupus nephritis. Urinary TWEAK is very specific for lupus nephritis and is correlated with the degree of kidney damage, but it is not sufficiently sensitive to predict episodes of nephritis. On the other hand, MCP-1 and NGAL are specific to kidney activity and can predict episodes of nephritis, but they have not been described as prognostic markers for the evolution of lupus. None of the three potential urinary markers has been related to kidney regeneration, nor to the remission of the disease in longitudinal studies (8).

Hence, despite the efforts made, there is still a need to find new markers with a sample which is easy to obtain and has a prognostic nature able to orient medical professionals in the best treatment.

Neuropilins (NRP-1 and NRP-2) are transmembrane proteins which interact with class-3 semaphorins, with members of the vascular endothelial growth factor (VEGF) family and ligands such as hepatocyte growth factor, platelet growth factor, “transforming growth factor-b1” (TGF-b1), and the fibroblast growth factor (FGF2) (10). They are thus co-receptors of VEGF and co-receptors of angiogenic factors such as HGF, and may increase the angiogenic activity of the latter. Neuropilins have been involved in different biological processes such as tumour growth and/or vascularisation, as mediators in primary immune response, or as inductors in regeneration and repair processes (10).

Meanwhile, vascular endothelial growth factor (VEGF) is an important cytokine involved in angiogenesis, chemotaxis and vascular permeability (11), has a protecting role in the preservation and function of organs, probably by maintaining cell function and the integrity of the vascular endothelium (12), and induces morphogenesis of kidney epithelial cells in dependent NRP-1 form (13).

The role of NRP-1 and VEGF in the pathogenesis of lupus nephritis has not been clarified. It has been suggested that high levels of VEGF have an important protective role in renal pathology (14, 15) and particularly in SLE (12, 16-18) where low levels of VEGF in urine have been associated with serious repercussions and a poor prognosis (17, 19); these works highlight the role of VEGF as prognosis of kidney disease, but they do not distinguish between the different kidney pathologies (20). Other studies carried out in patients with lupus nephritis have also shown an increase in NRP-1 in the kidneys in samples from biopsies on patients with SLE but not in other illnesses (21). Given that NRP-1 is a functional receptor of VEGF, it is thought that its increase means there is stimulation of the protective effect of VEGF in glomerular endothelial cells, helping to prevent damage and apoptosis (17) and specifically SLE, as it is found in high levels in both renal biopsy samples (17) and in synovial fluid (22). However, to date, the studies carried out on NRP-1 do not identify it as a prognostic marker of the lupus disease nor, particularly, of the development of the disease in the kidneys; in addition, up till now kidney biopsies has been used as samples, that is an invasive test which is not exempt from complications in the long term, and which makes it very difficult to identify the levels of NRP-1 rapidly, and also to monitor patients and the effectiveness of their treatment on kidney regeneration.

The evaluation of a certain marker in urine, able to indicate the prognosis for the disease at the moment the diagnosis is made and throughout its treatment, would allow the treatment to be more individual and lead to a great improvement in the morbidity and the mortality of these patients and, consequently, in their prognosis and quality of life.

### EXPLANATION OF THE INVENTION

### Brief Explanation of the Invention

A first object of the invention refers to the use of the Neuropilin 1 gene, NRP1, and its expression products as a prognostic marker of the development of nephritis, and more preferably lupus nephritis.

Another object of this invention refers to a method of quantification of the levels of NRP-1 as prognostic marker for the development of nephritis, preferably lupus nephritis.

A further object of the invention refers to a first method of the invention which allows quantification of the levels of nucleic acid in a urine sample, preferably mRNA. Preferably, said method is the RT-PCR, and even more preferably the qRT-PCR.

Another object of the invention refers to a second method of the invention, which allows detection of the levels of the protein NRP-1 in a sample, preferably in a urine sample.

A further object of the invention refers to an immunoassay which allows the detection of the NRP-1 protein in a sample, and more preferably to an ELISA.

A further object of the invention refers to a method of mass spectrophotometry which allows the quantification of NRP-1 in a sample, and more preferably the SELDI-TOD, or the MALDI-TOF.

Another particular object of this invention is related to a kit for nephritis prognosis, useful for the embodiment of the first or second method of the invention.

Thus, another object of the invention is the use of the first and second methods of the invention, or of the invention kit, to evaluate the development in urological patients of nephritis or lupus nephritis, or to evaluate the effect of a drug or drug candidate in urological patients with nephritis or lupus nephritis.

### DETAILED DESCRIPTION OF THE INVENTION

The inventors of this invention have identified that the levels of expression of NRP-1 in urine as a biomarker associated with nephritis, and particularly with lupus nephritis, and that it is a prognostic indicator of recovery. The use of this marker in urine supposes a substantial improvement on the current state of the art, which only uses kidney biopsy, a very invasive technique with long-term problems for patients, to monitor how the kidney is affected in lupus nephritis. The changes in NRP-1 can also be useful for overseeing the stages of the illness, its progression and the toxicity or pharmacological effectiveness of the clinical treatments to control said disease.

Thus, a first object of the invention is the use of NRP1 as a prognostic marker of the development of nephritis, and more preferably lupus nephritis.

Another particular object of this invention is the method of quantification of the levels of NRP-1 which can be used as a prognostic marker for the development of nephritis, preferably lupus nephritis, hereinafter the method of the invention. Thus, for example, a sample of urine from a patient can be analysed using any of the methods described here, or by any other method known to the experts in the art, to quantify the levels of expression of NRP-1 and compare them with base levels of expression obtained in patients not affected by the complaint, or by comparing the levels of expression in two patients to deduce the best/worst prognosis of them. The comparison of the NRP-1 levels is carried out by the researcher or by the person responsible for the diagnosis, or with the help of a computer and database.

The term “biomarker” or “biomarkers” refers to a molecule, such as a protein for example, which indicates a particular pathological state. An effective biomarker for lupus nephritis is typically a molecule which is secreted or excreted or, for example, eliminated in the urine through the kidneys.

The term “prognosis” as used in this invention refers to the procedure which establishes a prediction of the events which will occur in the development or course of an illness, preferably a kidney disease with an inflammatory component, more preferably lupus nephritis including, though not limited to, the predisposition to suffer from said illness or the capacity of response to a certain treatment.

The term “prediction” refers here to a method for forming a prognosis, in which a medically trained person analyses the information from one or more biomarkers.

The term “nephritis” or nephropathy refers to a kidney disease characterised by inflammation of the kidneys. Nephritis is generally the result of a diffuse inflammatory process which has as its basis an immunological process; said process begins when a foreign substance (antigen) enters the bloodstream and initiates the organism's defence mechanisms, including the production of antibodies. The union of the antigen with the antibody forms a soluble antigen-antibody complex which circulates through the organism and, if deposited in the tissues, generates inflammatory lesions; when this occurs in the kidney, it generates nephritis.

The term “lupus nephritis” refers to a kidney complaint which is a complication of the autoimmune illness, systemic lupus erythematosus (or SLE). It is characterised by the appearance of inflammatory lesions in the kidney, which leads to a worsening in kidney activity, which may include damage to the glomerules and the progressive loss of kidney function, which may eventually require dialysis or a kidney transplant.

Systemic Lupus Erythematosus (SLE) is an autoimmune disease, which means that the body's immune system erroneously attacks healthy tissue. This leads to the appearance of prolonged (chronic) inflammation. Some people with SLE have abnormal deposits in the kidney cells, which leads to the appearance of a complaint called lupus nephritis. Patients with this complaint may eventually suffer from renal insufficiency and require dialysis or a kidney transplant.

The term “biological sample”, as used here, refers to any substance derived from a living organism. For example, a sample may be derived from the blood, a urine sample, a serum sample, a plasma sample, or a sample of full blood. Alternatively, a sample can be derived from a tissue collected, for example, by means of a biopsy. Such a tissue sample might include, for example, kidney tissue, vascular tissue and/or heart tissue. A biological sample can also include bodily fluids including, but not limited to, urine, saliva or sweat.

The term “assay” generally means an analysis (including an analysis by SDS PAGE, ELISA, Western Blot) carried out on a sample to determine the presence of a substance and/or the quantity or the level of the substance in the sample. An assay can thus be carried out, for example, to determine the level of a biomarker of nephritis, and particularly lupus nephritis, in a biological sample.

The term “outbreak” or “kidney outbreak” refers to a significant increase in the inflammation in the orientation kidney of a subject who is already experiencing active lupus nephritis, which may result in a significant and reproducible increase in serum creatinine, proteinuria and/or hematuria, and a reduction in the kidney function.

The expressions “normal, healthy subject” or “healthy control” mean a person who is not experiencing reduced kidney function, such as acute or chronic kidney disease, acute kidney inflammation, acute infection, or another condition or disease which may increase the level of kidney biomarkers such as the excretion of protein or creatinine.

The expression “worsening of activity of the kidney disease” refers to a reduction in kidney activity caused by the disease, such as the worsening of lupus nephritis, a kidney outbreak, an additional reduction in kidney function, and in general refers to a subject diagnosed with SLE or another disease with an autoimmune or inflammatory basis.

The term “Neuropilin-1” or “NRP-1” refers to a protein codified by the human gene “nrp-1” with sequence NG_030328. (NCBI Reference) which codifies for a protein with sequence O14786 (UniProtKB Reference) and to, at least, 5 alternative transcripts (NP_001019799.1, NP_001019800.1, NP_001231901.1 NP_001231902.1 NP_003864.4).

The expression of NRP-1 is seen in both the level of messenger ribonucleic acid (mRNA) and of its product, which is the protein NRP-1. It has been seen that in patients suffering from lupus nephritis the increase in NRP-1 present in urine compared with the detected levels of NRP-1 in healthy controls determined by the levels of mRNA is associated with the progress of the effect in the kidneys in patients with lupus nephritis. Given that both lupus nephritis and nephritis have as an underlying basis the appearance of an inflammatory process in the kidney, the measurement of the mRNA levels in patients suffering from non-lupus nephritis will also be indicative of its prognosis value.

Thus, in a particular object of the invention, the detection method for levels of NRP-1 in urine uses the measurement of the levels of nucleic acid in a sample, preferably in urine, preferably mRNA NRP-1, called the first method of the invention. This is achieved through the hybridization of the nucleic acid in the urine with oligonucleotide probes which are specific for the NRP-1 gene. The technique used may be for illustrative purposes, and without limiting the scope of the invention, belonging to the following group: Northern blot analysis, polymerase chain reaction (PCR), reverse transcription in combination with the polymerase chain reaction (RT-PCR) in real time, reverse transcription in combination with the ligase chain reaction, hybridization or microarrays.

Samples of nucleic acids can be prepared using any of the methods and assays in this invention, or by any other method available in the state of the art. RNA isolation methods are well known by the experts in the art and include, though are not limited to, purification using oligo(dT) (associated with sepharose columns or magnetic particles, for example), and liquid-liquid biochemical extraction methods such as thiocyanate of guanidine-phenol-chloroform extraction or extraction with phenol-chloroform. An expert in the art will appreciate that it is desirable to include in the RNA isolation treatment chemical compounds which are useful for inhibiting or destroying RNases present in the homogenates before these can be used (RNAse inhibitors). The isolated nucleic acids include isolated mRNA, but also cDNA synthesised from a sample of mRNA isolated from a cell or tissue of interest. These samples also include DNA amplified from the cDNA, and an RNA transcribed from the amplified DNA.

The cDNA synthesis can be obtained using reverse transcription, generally combined with a DNA amplification to obtain a greater quantity of cDNA for analysis, in a technique known as RT-PCR.

The term “reverse transcription” refers to the synthesis of complementary DNA from an RNA template.

The term “amplification” refers to the increase in the number of copies of a nucleic acid template; the amplification generally takes place using a PCR.

The term “nucleic acid template” or “template” as used in this description refers to a molecule of single-chain or double-chain nucleic acid which is going to be reverse transcripted and/or amplified.

A method of reverse transcription of a nucleic acid template, preferably mRNA, generally comprises the following stages:

a) mixing the nucleic acid template with an enzyme with reverse transcriptase activity, and

b) incubating the mixture from step (a) in conditions allowing the synthesis of complementary DNA to the nucleic acid template.

A method of reverse transcription and amplification, RT-PCR, of a nucleic acid template, preferably mRNA, generally comprises the following stages:

a) mixing said nucleic acid with an enzyme with reverse transcriptase activity and with, at least, a polymerase DNA dependent on DNA, and

b) incubating the mixture from step (a) in conditions allowing the amplification of complementary DNA to the nucleic acid template.

The expression “conditions allowing the synthesis of complementary DNA” refers to the conditions in which the incorporation of the nucleotides to a nascent DNA may take place via the complementary nature of bases with the nucleic acid template.

Generally, the conditions in which DNA synthesis takes place include: (a) placing said nucleic acid template in contact with a reverse transcriptase in a mixture which also includes a primer, a bivalent cation, for example Mg2+, and nucleotides, and (b) subjecting said mixture to a sufficient temperature for a DNA polymerase to initiate the incorporation of the nucleotides to the primer using complementarity of bases with the nucleic acid template, and give rise to a population of complementary DNA molecules of different size. The separation of said population of complementary DNA molecules allows the sequence of nucleotides in the nucleic acid template to be determined.

The detection of the levels of a nucleic acid in the sample may be carried out by any of the methods known in the state of the art. In a preferred embodiment, the detection method involves the hybridization of the nucleic acids through contact between a probe and the target nucleic acid in conditions in which the probe and its complementary target can form stable hybrid duplexes by means of pairing of complementary bases. The methods of hybridization of nucleic acids are well known in the state of the art. In a preferred embodiment, the probes are marked with a fluorescent molecule. The hybridized nucleic acids are detected by detecting one or more labels on the nucleic acids in the sample and the probes. The labels can be incorporated by any of the methods known by the experts in the art.

Commonly used marking labels include, but are not limited to, biotin, fluorescent molecules, radioactive molecules, chromogenic substrates, chemical-luminescent markers, enzymes and similar. The methods for biotinylation of nucleic acids are well known in the art, as are the methods for introducing fluorescent molecules and radioactive molecules in oligonucleotides and nucleotides.

In the state of the art, there are known methods such as qRT-PCR (also called quantitative or real-time RT-PCR) which allows a nucleic acid to be reverse transcribed, amplified and quantified in a single step. In general, qRT-PCR comprises the following steps:

a) mixing the isolated nucleic acid, for example mRNA, with an enzyme with reverse transcriptase activity and with, at least, a polymerase DNA dependent on DNA,

b) incubating the mixture in step (a) in conditions which allow the amplification of the complementary DNA to the nucleic acid template using a primer, and

c) carrying out the hybridization in the presence of a fluorescent molecule which allows quantification of the cDNA generated with a specific detector.

The methods for detection of the quantity of nucleic acid produced in the qRT-PCR use either 1) non-specific fluorochromes, which detect the exponential generation of double-stranded DNA using a fluorochrome which attaches non-specifically to the former, such as, for example, SBR Green; or 2) specific probes which use at least one fluorescently marked oligonucleotide. Typically, this probe is attached to two fluorochromes and hybrids in the intermediate area between the direct primer (forward) and the inverse (reverse); in other words, in the amplicon. In this way, when the probe is intact, they present a fluorescence resonance energy transfer (FRET). Said FRET does not occur when the fluorochromes are distant, because of the degradation of the probe through the 5′-3′ exonuclease activity of the polymerase DNA, or because of the physical separation of the fluorochromes through a change in the configuration of the probe. This allows the change in the pattern of fluorescence to be monitored and the level of amplification of the gene to be deduced.

In a particular embodiment of the invention, the first method of the invention detects the levels of mRNA NRP-1 in a sample, preferably urine, using the qRT-PCR, and even more preferably, the first method of the invention comprises the following steps:
a) mixing the isolated mRNA from a sample with an enzyme with reverse transcriptase activity and with, at least, a polymerase DNA dependent on DNA,
b) incubating the mixture in step (a) in conditions which allow the amplification of the complementary DNA to the nucleic acid template using a primer, and
c) carrying out the detection in the presence of a fluorescent molecule which allows quantification of the cDNA generated with a specific detector.

Even more preferably, the fluorescent molecule used in step c) is SYBR Green or TaqMan.

In another particular object of the invention, the method for detection of the levels of NRP-1 in a sample uses the measurement of the levels of NRP-1 protein, preferably in a urine sample, henceforth referred to as the second method of the invention. The methods for detection and quantification of a protein include immunoassays and mass spectrometry analysis. Both methods allow the simultaneous detection of various proteins of interest to be combined.

In a particular embodiment, the second method of the invention comprises detection of the levels of NRP-1 protein using an immunoassay. In general, immunoassays involve the combining of NRP-1 with an anti-NRP-1 antibody. The presence and the amount of union indicate the presence and quantity of NRP-1 present in the sample. Examples of immunoassays include, but are not limited to, ELISA, radio-immunoassays, and immunoblots, which are well-known in the art. The antibody may be polyclonal or monoclonal, and is preferably marked for easy detection. The labels may be, but are not limited to, biotin, fluorescent molecules, radioactive molecules, chromogenic substrates, chemical luminescence and enzymes. In order to quantify the amount of NRP-1 present in the sample, the immunoassay must allow comparison between the levels of NRP-1 and a standard or control sample; quantification methods associated with immunoassays are very well-known by experts in the state of the art, and amongst these we might mention, for example, densitometry, spectrophotometry, fluorometry, or CBA (BD™ Cytometric Bead Array).

In a preferred embodiment, the second method of the invention is an ELISA (Enzyme-Linked ImmunoSorbent Assay), a technique in which an immobilised antigen is detected by means of an antibody linked to an enzyme capable of generating a detectable product such as a change of colour or any other type. The appearance of colour allows the antigen in the sample to be measured indirectly by spectrophotometry. In the state of the art, various types of ELISA strategies are known, such as the direct ELISA, indirect ELISA or the ELISA Sandwich.

In the direct ELISA, a support is prepared and covered with the solutions in which it is suspected that the antigen is to be found. They are incubated with marked antibodies which indicate the presence of antigen in the solution analysed. In the indirect ELISA, the initial support is prepared in the same way as for the direct ELISA. The detection system uses two antibodies: a primary one against the antigen and a secondary one marked against the primary one. There is greater sensitivity in the detection because of an amplification of signal due to the union of two or more secondary antibodies for each primary. This assay also allows the use of a same marked secondary and a same enzyme system, which allows quantification of a wide variety of antigens.

The “sandwich” ELISA is an assay in which the ELISA support is covered with a first anti-antigen antibody. Subsequently, the problem sample containing the antigen is applied, and the antigen will be retained and captured by the first antibody. In a subsequent incubation, a second anti-antigen antibody is used, marked with some kind of marking label. In this way, each molecule of antigen will be attached to an antibody in the base which retains it, and a second antibody, at least, which marks it.

The term “support” refers to a material which allows the joining of the antibodies and serves as a physical support for the assay. The most commonly used types of support used in ELISA are plastic well plates, although nano-particles can also be used.

Commonly used marking labels for antibodies include, but are not limited to, biotin, fluorescent molecules, radioactive molecules, chromogenic substrates, chemical-luminescent markers, enzymes and similar. The methods for marking antibodies are well-known in the art. The marking method used determines the mode of detection of the signal, be it spectrometry, densitometry, luminometry, fluorometry, etc.

The term “chromogenic substrate” refers to a molecule which, after undergoing a process of enzymatic alteration, changes its spectral properties. In the context of this invention, it refers to the substrate which, when added to the ELISA, produces a measurable and quantifiable colorimetric signal. The choice of chromogenic substrate depends on the detection method used: the most well-known are ABTS or (2,2′-azino-bis(3-ethylbenzothiazoline-6-sulphonic acid) or TMB (3,3′,5,5′-Tetramethylbenzidine) when the enzyme coupled to the reaction is peroxidase, or p-Nitrophenylphosphate (p-NPP) when the enzyme coupled to the reaction is alkaline phosphatase.

A concrete example might be the capture of NRP-1 on the biological sample in a Sandwich-type ELISA assay, in which an immobilised anti-NRP-1 monoclonal antibody is followed by detection with a polyclonal antibody marked with anti-NRP-1 biotin. In this system, the wells in a plate with multiple wells are covered with the monoclonal antibody and are blocked with an appropriate blocking buffer; the urine samples are then added to the wells and incubated for the capture of NRP-1 by the monoclonal antibody. The polyclonal detection antibody is then added to the plate and, finally, a conjugate of streptavidin phosphatase to obtain colour through the appropriate substrate. The intensity of the colour obtained can be quantified by using a spectrophotometer suitable for the chromogen used.

Thus, in an even more preferred embodiment, the second method of the invention comprises the following steps:
a) immobilisation of a monoclonal anti-NRP-1 antibody to a support,
b) incubation with the sample and capture of the protein of interest,
c) incubation with a polyclonal antibody marked with anti-NRP-1 biotin,
d) attachment of a streptavidin alkaline phosphatase conjugate
e) Revealing of the enzymatic reaction with a chromogenic substrate, e.g. p-nitrophenyl phosphate (p-NPP)
f) Analysis of the optical density using spectrophotometry at the wavelength appropriate for the chromogenic substrate used.

Alternatively, after step e) an additional step can be carried out with the addition of a chemical component which stops the enzymatic reaction. Another possible way of analysing and quantifying the amount of a certain protein in a sample is by Mass Spectrometry (MS) analysis; this is an experimental technique which allows the measurement of ions derived from molecules, separating the molecules or the fragments thereof depending on their mass-charge (m/z) ratio. In analysis of complex samples, such as urine, this has been combined with methods such as chromatography, like gas chromatography (GC/MS) or liquid chromatography (LC/MS), to allow a separation between the different components of the sample. In the case of protein mixtures, the techniques already known in the state of the art are, for example, MALDI-TOF (where MALDI comes from Matrix-Assisted Laser Desorption/Ionization and TOF from Time-of-Flight), SELDI-TOF (Surface-enhanced laser desorption/ionization and Time-Of-Flight), and ESI-MS (electrospray ionization coupled with Mass Spectrometry).

Thus, in another preferred embodiment, the second method of the invention is the SELDI-TOF, or the MALDI-TOF.

Another particular object of this invention is related to a kit for nephritis prognosis, preferably lupus nephritis, hereinafter referred to as the useful invention kit for the embodiment of the method of the invention. Preferably, the prognostic kit comprises using the detection of NRP-1 alone, or in combination with other markers for better evaluation of present state and development of the disease in an individual.

A particular embodiment of the invention is the kit which allows the first method of the invention to be carried out, called the first kit of the invention, and which allows a quantification of the amount of NRP-1 mRNA in a biological sample, preferably in urine, and which comprises:
a) a reverse transcriptase, and
b) at least one element from the list which comprises:
   i) a buffer,
   ii) a primer,
   iii) a DNA-dependent DNA polymerase, and
   iv) a nucleotide.

Another particular embodiment of this invention is the kit which allows the second method of the invention to be carried out, called the second kit of the invention, and which allows quantification of the amount of NRP-1 protein in a biological sample, preferably in urine, and which comprises:
a) an anti-NRP1 monoclonal antibody,
b) an anti-NRP1 polyclonal antibody, preferably conjugated with biotin,
c) a conjugate of streptavidin alkaline phosphatise, and
d) a chromogenic substrate like p-NPP.

In this system, the support is a plate with multiple wells covered with a monoclonal antibody; subsequently, the samples of urine are added to the wells and incubated for the capture of NRP-1 by the monoclonal antibody. The polyclonal detection antibody is then added to the plate and, finally, a conjugate of streptavidin alkaline phosphatase to obtain colour through the appropriate substrate. The intensity of the colour obtained can be quantified by using a spectrophotometer suitable for the chromogen used.

Thus, in accordance with this invention, the levels of NRP-1 can also be used as markers to evaluate the effects of a drug or a drug candidate in urological patients with lupus nephritis. In addition, an expert in the art will be aware of the application of said methods to urine samples from non-human mammals, which are experimentation models for lupus nephritis.

A patient is treated with a drug candidate while the progression of the disease is monitored over time. This procedure comprises the treatment of the patient with an agent, the obtaining of a urine sample from the patient, the determination of the levels of NRP-1 in the urine and comparing said levels over time to determine the effect of the agent on the progression of the disease.

Thus, another object of the invention is the use of the first and second methods of the invention, or of the invention kit, to evaluate the development in urological patients of nephritis or lupus nephritis. In a particular embodiment, said use is employed to evaluate the effect of a drug or drug candidate on urological patients with nephritis or lupus nephritis.

Throughout the description and the claims, the word “comprises” and its variants are not intended to exclude other technical features, additives, components or steps. For experts in the matter, other objects, advantages and characteristics of the invention will be deduced in part from the description and in part from the practice of the invention. The following examples and figures are provided for illustrative purposes, and they are not intended to be limitations on this invention.

### DESCRIPTION OF THE FIGURES

FIG. 1. Increase in the expression of NRP-1 at the moment of diagnosis in the group of patients who would be cured (scale from 0 to 600000) compared to the group which would not be cured (scale from 0 to 800), where this is statistically significant (p<0.0001).

FIG. 2. Expression in urine of NRP-1 at the moment of the diagnosis and after a year with standard treatment (corticoids and immunosuppressive treatment) of lupus nephritis in patients who were cured completely. The results indicate a reduction in the expression of NRP-1 after a year of treatment compared to the value at the moment of diagnosis, where this is statistically significant (p<0.0001).

FIG. 3. Expression in urine of NRP-1 at the moment of diagnosis and upon completion of one year of treatment in patients who are not cured. The results indicate a significant increase in the expression of NRP-1 after a year of treatment compared to the value at the moment of diagnosis, where this is statistically significant (p<0.0001).

FIG. 4. ROC curve to calculate the sensitivity and the specificity of the use of the urinary expression of Neuropilin-1 and of the anti-DNA antibodies as biomarkers in prognosis of cure at the moment of diagnosis in patients with lupus nephritis, after the administration of the treatment.

FIG. 5. Levels of NRP-1 in urine evaluated by ELISA (ng/ml) at the moment of diagnosis of the outbreak of nephritis (time 1) and after 15 months of standard treatment with corticoids and immunosuppressant drugs (time 2) in patients who achieved remission of the disease (remission) and in those who did not achieve it (no remission). The symbol “+” indicates p<0.001 v control. The asterisk symbol (*) indicates p<0.001 v no remission.

FIG. 6. NRP-1/creatinine quotient in urine at the moment of diagnosis of the outbreak of nephritis (time 1) and after 15 months of standard treatment with corticoids and immunosuppressant drugs (time 2) in patients who achieved remission of the disease (remission) and in those who did not achieve it (no remission). The symbol “+” indicates p<0.001 v control. The asterisk symbol (*) indicates p<0.001 v no remission.

### EMBODIMENT OF THE INVENTION

### Example 1

Of a cohort of patients with SLE and kidney problems as described in (23), monitored during the evolution of their disease, 24 patients were selected who achieved a complete cure after treatment and 24 who did not achieve it; all were monitored throughout the process in the Vall d'Hebrón Hospital. In both groups the same treatment was used to induce remission of the lupus nephritis (corticoids and mycophenolate).

A sample of urine was taken from each of the patients, obtained on the day of diagnosis and after one year of treatment. The sample was centrifuged at 3900 rpm (4° C.) for 30 minutes, to obtain a pellet of supernatant, which was stored at −80° C. until subsequent analysis.

From the pellet, using the process of reverse transcription of ribonucleic acid (RNA), complementary DNA (cDNA) was obtained; subsequently, the expression of neuropilin-1 was quantified using a real time quantitative PCR (RT-Q-PCR). The primers were as follows:

Fw
5′-CACAGTGGAACAGGTGATGACTTC-3′
Rv
5′-AACCATATGTTGGAAACTCTGATTGT-3′

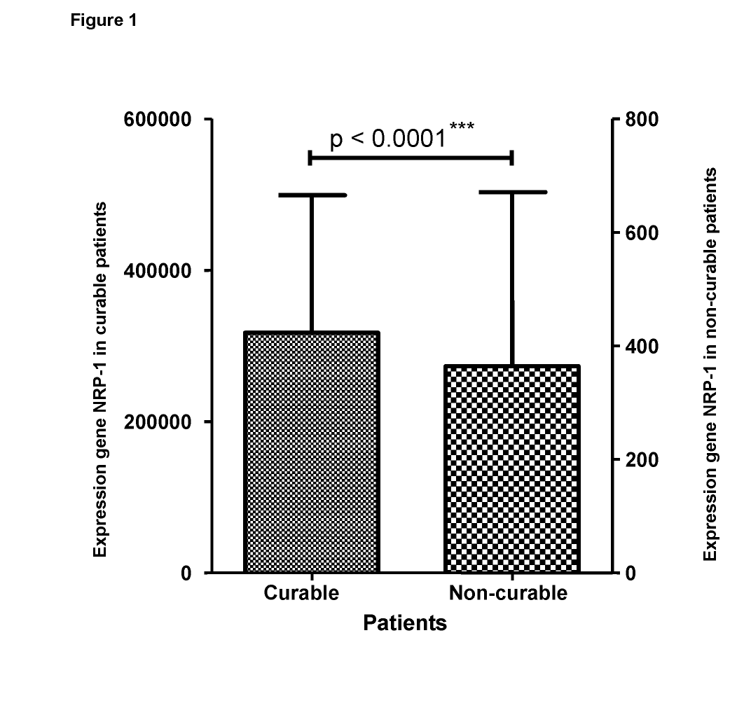

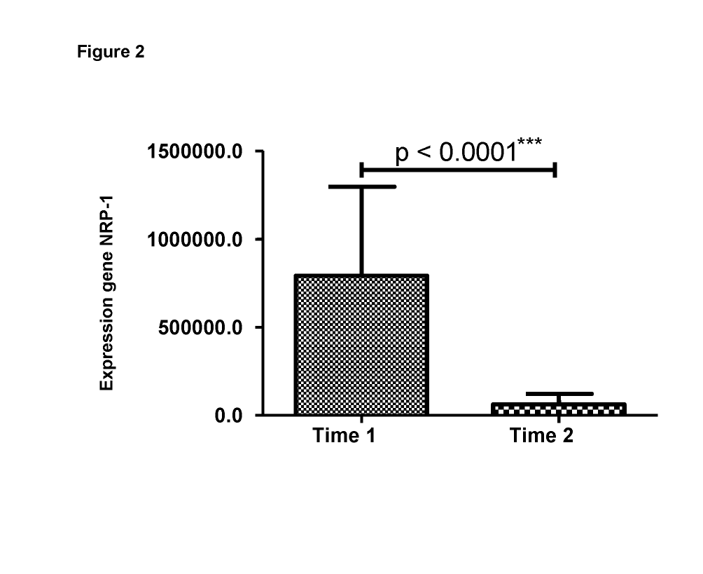

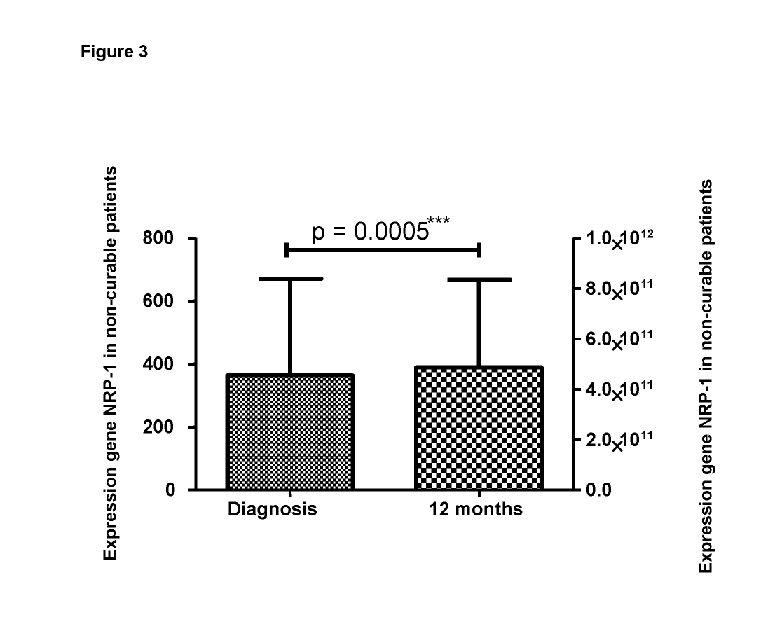

An increase in the expression of the NRP-1 gene in the mRNA was observed at the moment of diagnosis in the group of patients which would achieve cure compared to the group which would not achieve it (FIG. 1), where the expression is statistically significant (p<0.0001). In addition, the NRP-1 gene was quantified in both groups when the year of treatment was completed: a reduction in its expression was seen in the group which was completely cured, with the expression being statistically significant (p<0.0001) (FIG. 2), with a significant increase (p<0.0005) in the group which was not cured (FIG. 3).

Correlations were also made between parameters used habitually in the monitoring of these patients (urinary SLEDAI, proteinuria (mg/dL and in 24 hours), sediment in urine, creatinine clearance and haemoglobin and leukocyte values in blood) using the values of our patients one year after the start of the study and the values for the gene expression of neuropilin-1 at the moment of diagnosis. The only correlation found was a significant inverse correlation between the values for gene expression of neuropilin-1 and the values of proteinuria in mg/dL (r=−0.4351, p=0.0336) and in 24 hours (r=−0.4955, p=0.0190).

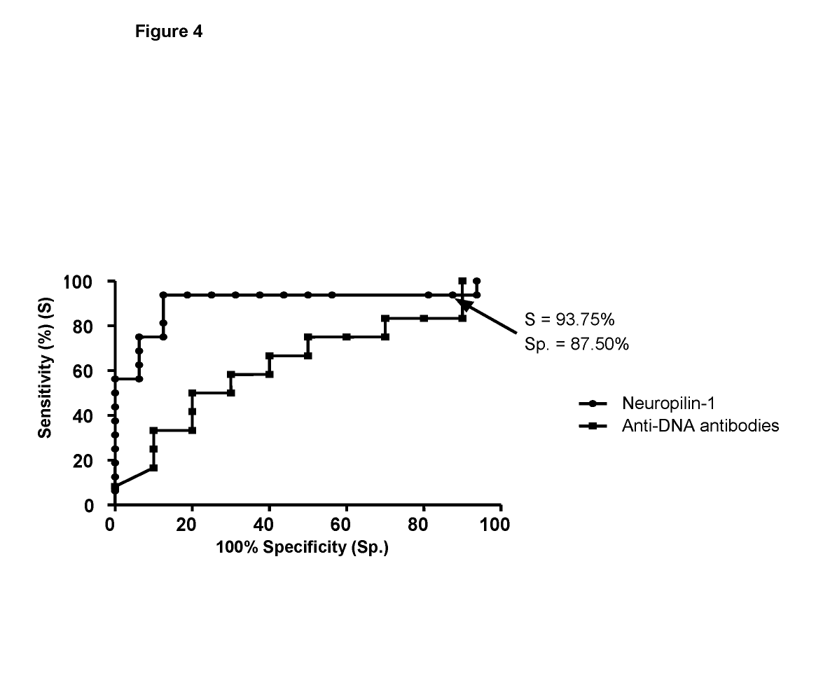

To calculate the specificity and sensitivity of the expression of neuropilin-1 to predict the prognosis of lupus nephritis at the moment of its diagnosis, an ROC curve was created which showed that for values >244 it has a sensitivity of 93.75% and a specificity of 87.50%, which are clearly higher than the anti-DNA antibodies in the same clinical situation (FIG. 4).

The results obtained in our research indicate that the biomarker which is the object of our patent predicts at the moment of diagnosis the prognosis of lupus nephritis and that it does so in a much better way than the clinical parameters used in usual clinical practice.

### Example 2

Of a cohort of patients with SLE and kidney problems, monitored during the evolution of their disease, 24 patients were selected who achieved a complete cure after treatment and 24 who did not achieve it; all were monitored throughout the process in the Vall d'Hebrón Hospital. In both groups the same treatment was used to induce remission of the lupus nephritis (corticoids and mycophenolate).

The amount of neuropilin-1 (NRP-1) in urine was evaluated using a commercial detection kit (Cloud-Clone Corp, Houston, USA). The kit is an enzymatic immunoassay sandwich for the quantitative measurement in vitro of NRP-1 in human serum, plasma, tissue homogenates, urine or other biological fluids. The assay was carried out according to the manufacturer's instructions: 100 μl of standard or sample (dilution 1:10-1:100) was added to each well in the plate. After 2 h of incubation at 37° C., 100 μl of detection reactant was added and incubated for 1 hour at 37° C. After 5 washes with washing solution. 100 μl of detection reactant were added and incubated for 30 minutes at 37° C. The plate was washed five times, 90 μl of substrate solution was added and after 20 minutes at 37° C. (protected from the light), the reaction was stopped with 50 μl of stopping solution and the absorbency was measured at 450 nm immediately. All the determinations were made in triplicate and with standard dilutions (curve between 20 and 0.312 ng/ml).

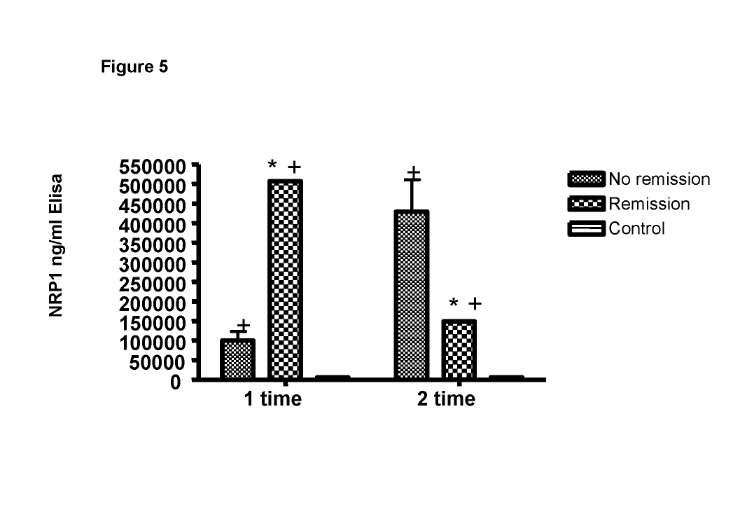

As shown in FIG. 5, the results indicate a significant increase in the levels of NRP-1 in time 1 (507007±83699 ng/ml) in the group of patients with the disease in remission, compared to the group of patients with no remission (100043±23702). In time 2, the results indicate a significant decrease in the levels of NRP-1 in the group of patients with remission (149283±24349 ng/ml), compared to the group of patients with no remission (429193±82003 ng/ml). Results expressed in average±error.

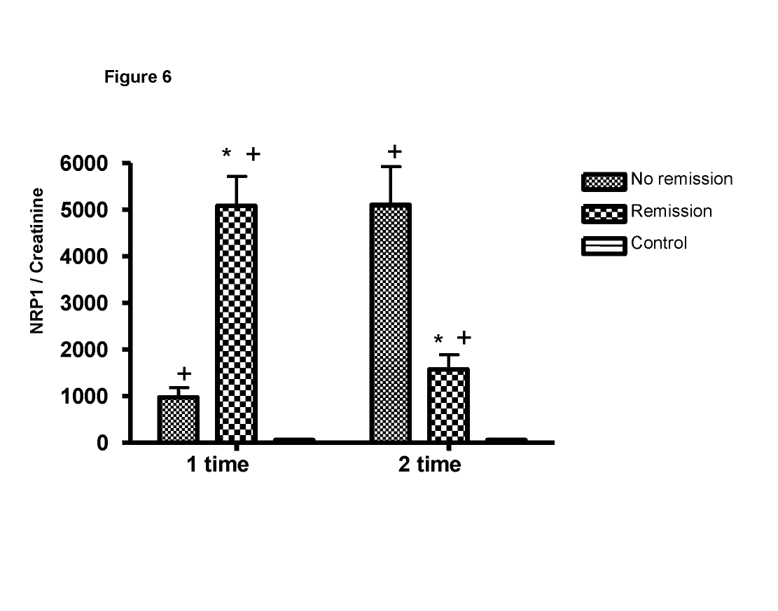

As shown in FIG. 6, the results indicate a significant increase in the levels of NRP-1 in time 1 (5082±628 average±em) in the group of patients with the disease in remission, compared to the group of patients with no remission (960±212). In time 2, the results indicate a significant drop in the levels of NRP-1 in the group of patients with remission (1572±−313), compared to the group of patients with no remission (5104±820). Results expressed in average±error.

### BIBLIOGRAPHY

1. Faurschou M, Starklint H, Halberg P, Jacobsen S. 2006. Prognostic factors in lupus nephritis: diagnostic and therapeutic delay increases the risk of terminal renal failure. J Rheumatol 33: 1563-9.
2. Rosner S, Ginzler E M, Diamond H S, Weiner M, Schlesinger M, Fries J F, Wasner C, Medsger T A, Jr., Ziegler G, Klippel J H, Hadler N M, Albert D A, Hess E V, Spencer-Green G, Grayzel A, Worth D, Hahn B H, Barnett E V. 1982. A multicenter study of outcome in systemic lupus erythematosus. II. Causes of death. Arthritis Rheum 25: 612-7.
3. Fiehn C. 2006. Early diagnosis and treatment in lupus nephritis: how we can influence the risk for terminal renal failure. J Rheumatol 33: 1464-6.
4. Swaak A J, Groenwold J, Bronsveld W. 1986. Predictive value of complement profiles and anti-dsDNA in systemic lupus erythematosus. Ann Rheum Dis 45: 359-66.
5. Zappitelli M, Duffy C M, Bernard C, Gupta I R. 2008. Evaluation of activity, chronicity and tubulointerstitial indices for childhood lupus nephritis. Pediatr Nephrol 23: 83-91
6. Cameron J S. 1999. Lupus nephritis. J Am Soc Nephrol 10: 413-24.
7. Austin H A, 3rd, Boumpas D T, Vaughan E M, Balow J E. 1994. Predicting renal outcomes in severe lupus nephritis: contributions of clinical and histologic data. Kidney Int 45: 544-50.
8. Mok C C. 2010. Biomarkers for lupus nephritis: a critical appraisal. J Biomed Biotechnol 2010: 638413.
9. Manoharan A, Madaio M P. 2010. Biomarkers in lupus nephritis. Rheum Dis Clin North Am 36: 131-43, ix.
10. Schramek H, Sarkozi R, Lauterberg C, Kronbichler A, Pirklbauer M, Albrecht R, Noppert S J, Perco P, Rudnicki M, Strutz F M, Mayer G. 2009. Neuropilin-1 and neuropilin-2 are differentially expressed in human proteinuric nephropathies and cytokine-stimulated proximal tubular cells. Lab Invest 89: 1304-16
11. Nieves B J, D'Amore P A, Bryan B A. 2009. The function of vascular endothelial growth factor. Biofactors 35: 332-7.
12. Feliers D. 2009. Vascular endothelial growth factor as a prognostic marker of lupus nephritis. Kidney Int 75: 1251-3.
13. Karihaloo A, Karumanchi S A, Cantley W L, Venkatesha S, Cantley L G, Kale S. 2005. Vascular endothelial growth factor induces branching morphogenesis/tubulogenesis in renal epithelial cells in a neuropilin-dependent fashion. Mol Cell Biol 25: 7441-8.
14. Shulman K, Rosen S, Tognazzi K, Manseau E J, Brown L F. 1996. Expression of vascular permeability factor (VPF/VEGF) is altered in many glomerular diseases. J Am Soc Nephrol 7: 661-6.
15. Kang D H, Joly A H, Oh S W, Hugo C, Kerjaschki D, Gordon K L, Mazzali M, Jefferson J A, Hughes J, Madsen K M, Schreiner G F, Johnson R J. 2001. Impaired angiogenesis in the remnant kidney model: I. Potential role of vascular endothelial growth factor and thrombospondin-1. J Am Soc Nephrol 12: 1434-47.
16. Frieri M, Samih M A, Dzhindzhikhashvili M, Liu H, Balsam L, Rubinstein S. 2012. Toll-like receptor 9 and vascular endothelial growth factor levels in human kidneys from lupus nephritis patients. J Nephrol 25: 1041-6.
17. Avihingsanon Y, Benjachat T, Tassanarong A, Sodsai P, Kittikovit V, Hirankarn N. 2009. Decreased renal expression of vascular endothelial growth factor in lupus nephritis is associated with worse prognosis. Kidney Int 75: 1340-8.
18. Avihingsanon Y, Phumesin P, Benjachat T, Akkasilpa S, Kittikowit V, Praditpornsilpa K, Wongpiyabavorn J, Eiam-Ong S, Hemachudha T, Tungsanga K, Hirankarn N. 2006. Measurement of urinary chemokine and growth factor messenger RNAs: a noninvasive monitoring in lupus nephritis. Kidney Int 69: 747-53.
19. Wongpiyabovorn J, Hirankarn N, Ruchusatsawat K, Yooyongsatit S, Benjachat T, Avihingsanon Y. 2011. The association of single nucleotide polymorphism within vascular endothelial growth factor gene with systemic lupus erythematosus and lupus nephritis. Int J Immunogenet 38: 63-7.
20. Paydas S, Balal M, Tanriverdi K, Sertdemir Y, Baslamisli F. 2007. The relationship between the VEGF levels and VEGF mRNA expression and clinical course in different glomerulonephritis. Ren Fail 29: 779-84.
21. Vadasz Z, Haj T, Halasz K, Rosner I, Slobodin G, Attias D, Kessel A, Kessler O, Neufeld G, Toubi E. 2012. Semaphorin 3A is a marker for disease activity and a potential immunoregulator in systemic lupus erythematosus. Arthritis Res Ther 14: R146.
22. Takagawa S, Nakamura F, Kumagai K, Nagashima Y, Goshima Y, Saito T. 2013. Decreased Semaphorin3A expression correlates with disease activity and histological features of rheumatoid arthritis. BMC Musculoskelet Disord 14: 40.
23. Balada E, Castro-Marrero J, Pujol A P, Torres-Salido M T, Vilardell-Tarres M, Ordi-Ros J. 2011. Enhanced transcript levels of CD48 in CD4(+) T cells from systemic lupus erythematosus patients. Immunobiology 216: 1034-7.

1-19. (canceled)

20. A method for detecting and monitoring of a nephritis disease comprising:
collecting a subject sample from a subject suffering nephritis; and
detecting and determining the levels of one or more biomarkers in the subject sample, wherein the one or more biomarkers comprises an NRP-1 gene or expression products of the NRP-1 gene.

21. The method according to claim 20, wherein the nephritis disease is lupus nephritis.

22. The method according to claim 20, wherein the expression product is mRNA NRP-1.

23. The method according to claim 21, wherein the expression products mRNA NRP-1.

24. The method according to claim 22 or 23, wherein the levels of the one or more biomarkers are determined using RT-PCR or qRT-PCR.

25. The method according to claim 24, wherein qRT-PCR comprises the following steps:
a) mixing isolated mRNA from the subject sample with an enzyme having reverse transcriptase activity and with, at least, a polymerase DNA dependent on DNA
b) incubating the mixture in step (a) in conditions which allows the amplification of the complementary DNA to the nucleic acid template using a primer, and
c) carrying out the detection in the presence of a fluorescent molecule which allows quantification of the cDNA generated with a specific detector.

26. The method according to claim 25, wherein the fluorescent detection in step c) is SYBR Green.

27. The method according to claim 20, wherein the expression product is NRP-1 protein.

28. The method according to claim 21, wherein the expression product is NRP-1 protein.

29. The method according to claim 27 or 28, wherein the levels of the one or more biomarkers are determining using an immunoassay, mass spectrometry or ELISA.

30. The method according to claim 29, wherein ELISA comprises the following steps:
a) immobilisation of a monoclonal anti-NRP-1 antibody to a support,
b) incubation with the sample and capture of the protein NRP-1,
c) incubation with a polyclonal anti-NRP-1 antibody marked with biotin,
d) attachment of a streptavidin alkaline phosphatase conjugate,
e) revealing of the enzymatic reaction with a chromogenic substrate.
f) analysis of the optical density using spectrophotometry at the wavelength appropriate for the chromogenic substrate used.

31. The method according to claim 30, wherein the chromogenic substrate is p-nitrophenyl phosphate (p-NPP).

32. The method according to claim 29, wherein the levels of the one or more biomarkers are determined using SELDI-TOF or MALDI-TOF.

33. The method according to claim 20, wherein the subject sample is urine.

34. A diagnostic kit for detecting and monitoring nephritis or lupus nephritis in a subject sample according to the method of claim 22, wherein the kit comprises an assay for detecting the levels of NRP-1 gene or the expression products of the NRP-1 gene comprising:
a) a reverse transcriptase, and
b) at least one element selected from the group consisting of:
i) a buffer,
ii) a primer,
iii) a DNA-dependent DNA polymerase, and
iv) a nucleotide.

35. A diagnostic kit for detecting and monitoring nephritis or lupus nephritis in a subject sample according to the method of claim 27, wherein the kit comprises an assay for detecting the levels of NRP-1 protein, said diagnostic kit comprising:
a) an anti-NRP1 monoclonal antibody,
b) an anti-NRP1 polyclonal antibody,
c) a conjugate of streptavidin alkaline phosphatise, and
d) a chromogenic substrate.

36. A diagnostic kit according to claim 35, wherein the anti-NRP1 polyclonal antibody is conjugated with biotin.

37. The diagnostic kit according to claim 34, wherein the subject sample is urine.

38. The diagnostic kit according to claim 35, wherein the subject sample is urine.

39. A method of evaluating the development in urological patients with nephritis or lupus nephritis by using the kit according to claim 34.

40. A method of evaluating the development in urological patients with nephritis or lupus nephritis by using the diagnostic kit according to claim 35.

41. A method of evaluating the effectiveness of a drug or drug candidate in urological patients with nephritis or lupus nephritis by using the diagnostic kit according claim 34.

42. A method of evaluating the effectiveness of a drug or drug candidate in urological patients with nephritis or lupus nephritis by using the diagnostic kit according claim 35.


SEQUENCE LISTING (2 sequences)

SEQ ID NO: 1 - DNA, 24 bp, Artificial Sequence
  Synthetic Forward Primer Sequence
cacagtggaacaggtgatgacttc

SEQ ID NO: 2 - DNA, 26 bp, Artificial Sequence
  Synthetic Reverse Primer Sequence
aaccatatgttggaaactctgattgt